(19) United States
(12) Patent Application Publication
(10) Pub. No.: US 2018/0132806 A1
(43) Pub. Date: May 17, 2018

(54) X-RAY DETECTOR AND X-RAY IMAGING APPARATUS HAVING THE SAME

(71) Applicant: Samsung Electronics Co., Ltd., Gyeonggi-do (KR)

(72) Inventors: Won Jun Choi, Hwaseong-si (KR); Jung Min Kim, Seoul (KR); Jae Won Yoo, Suwon-si, Gyeonggi-do (KR); Jae Wook Lee, Seoul (KR); Hyun Ho Choi, Seongnam-si (KR)

(21) Appl. No.: 15/730,616

(22) Filed: Oct. 11, 2017

(30) Foreign Application Priority Data
Nov. 16, 2016 (KR) 10-2016-0152986

(51) Int. Cl. A61B 6/00; A61B 6/10

(52) U.S. Cl. CPC A61B 6/4283; A61B 6/4464; A61B 6/102

(57) ABSTRACT

Disclosed herein is an X-ray detector having impact resistance and an X-ray imaging apparatus including the same. The X-ray imaging apparatus includes an X-ray source configured to generate X-rays and emit the generated X-rays and an X-rays detector configured to detect the X-rays emitted by the X-ray source. The X-ray detector includes a main body, a sensor panel disposed in the main body to convert the X-rays emitted by the X-ray source into electrical signals, a middle block disposed in the main body to support the sensor panel and a buffer member extending between the main body and the middle block and having at least one bent portion.

### CROSS-REFERENCE TO RELATED APPLICATION AND CLAIM OF PRIORITY

This application claims the benefit of Korean Patent Application No. 10-2016-0152986, filed on Nov. 16, 2016 in the Korean Intellectual Property Office, the disclosure of which is incorporated herein by reference.

### TECHNICAL FIELD

Embodiments of the present disclosure relate to an X-ray detector and an X-ray imaging apparatus including the same, and more particularly, to an X-ray detector having impact resistance and an X-ray imaging apparatus including the same.

### BACKGROUND

X-ray imaging apparatuses are used to acquire internal images of a target object by using X-rays. X-ray imaging apparatuses may noninvasively acquire images of the inside of the target object by irradiating the target object with X-rays and detecting X-rays having passed through the target object. Thus, a medical X-ray imaging apparatus may be used for diagnosis of injuries or illnesses inside the target object which may not be confirmed by the external appearance.

An X-ray imaging apparatus may include an X-ray source configured to generate X-rays and emit the generated X-rays toward a target object and an X-ray detector configured to detect X-rays having passed through the target object. The X-ray source may be movably provided to image various parts of the target object. The X-ray detector may be used in a table mode in which the X-ray detector is mounted on an imaging table, in a stand mode in which the X-ray detector is mounted on an imaging stand, or in a portable mode in which the X-ray detector is not fixed to any position.

When an external impact is applied to the X-ray detector, performance of the X-ray detector may deteriorate. Particularly, when an external impact is directly transmitted to a vulnerable portion such as a sensor panel, the vulnerable portion may be damaged or break down resulting in deterioration of performance of the X-ray detector.

### SUMMARY

To address the above-discussed deficiencies, it is a primary object to provide an X-ray detector having a structure capable of preventing a sensor panel from being damaged by an external impact and an X-ray imaging apparatus including the same.

It is another aspect of the present disclosure to provide an X-ray detector having a structure capable of protecting a sensor panel from external deformation of the X-ray detector by an external force and an X-ray imaging apparatus including the same.

Additional aspects of the disclosure will be set forth in part in the description which follows and, in part, will be obvious from the description, or may be learned by practice of the disclosure.

In accordance with one aspect of the present disclosure, an X-ray imaging apparatus includes an X-ray source configured to generate X-rays and emit the generated X-rays and an X-rays detector configured to detect the X-rays emitted by the X-ray source. The X-ray detector includes a main body to define an appearance, a sensor panel disposed in the main body to convert the X-rays emitted by the X-ray source into electrical signals, a middle block disposed in the main body to support the sensor panel and a buffer member extending from at least one of the main body and the middle block to have at least one bent portion.

The main body includes a top frame disposed to allow the X-rays emitted by the X-ray source to be incident thereon and a side frame coupled to the top frame to define a side appearance of the X-ray detector. The middle block comprises an edge surface facing the side frame and the buffer member extends from at least one of the side frame and the edge surface.

The main body further includes a bottom frame disposed to face the top frame. The bottom frame and the side frame are integrally formed with each other.

The side frame and the middle block are coupled to each other via the buffer member.

The buffer member includes a fastening part to couple the side frame with the middle block.

The buffer member extends from the side frame to have the at least one bent portion. One end of the buffer member is fixedly coupled to the edge surface.

The buffer member extends from the edge surface to have the at least one bent portion. One end of the buffer member is fixedly coupled to an inner surface of the side frame.

The buffer member includes a first buffer member extending from the side frame to have at least one first bent portion and a second buffer member extending from the edge surface to have at least one second bent portion. One end of the first buffer member and one end of the second buffer member are fixedly coupled to each other.

The buffer member includes a first buffer member extending from the side frame to have at least one first bent portion and a second buffer member extending from the edge surface to have at least one second bent portion. The X-ray detector further includes an impact absorbing member disposed between one end of the first buffer member and one end of the second buffer member facing the one end of the first buffer member.

The X-ray detector further includes a circuit board configured to control operation of the X-ray detector. The circuit board is disposed in the main body to be spaced apart from the middle block.

The X-ray detector further includes a battery configured to supply power to the circuit board. The battery is disposed in the main body to be spaced apart from the middle block.

In accordance with one aspect of the present disclosure, an X-ray detector provided to detect X-rays includes a main body having an internal space, a middle block disposed in the internal space, a buffer member extending from at least one of the main body and the middle block to have at least one bent portion and configured to partition the internal space into a first space and a second space together with the middle block and a sensor panel disposed in the first space to convert the X-rays into electrical signals.

In accordance with one aspect of the present disclosure, an X-ray detector further includes a circuit board configured to control operation of the X-ray detector and disposed in the second space to be spaced apart from the middle block.

In accordance with one aspect of the present disclosure, an X-ray detector further includes a battery configured to supply power to the circuit board and disposed in the second space to be spaced apart from the middle block.

The main body includes a top frame configured to define the first space and a side frame extending in an alignment direction of the first space and the second space and coupled to the top frame. The middle block includes an edge surface facing the side frame. The buffer member extends from at least one of the side frame and the edge surface.

The main body further includes a bottom frame to define the second space. The bottom frame and the side frame are integrally formed with each other.

The side frame and the middle block are coupled to each other via the buffer member.

In accordance with one aspect of the present disclosure, an X-ray detector provided to detect X-rays includes a main body, a sensor panel disposed in the main body to convert the X-rays into electrical signals, a middle block disposed in the main body to support the sensor panel and an impact path disposed between the main body and the middle block to move an impact applied to the main body and comprising at least one bent portion to reduce the impact applied to the main body.

The main body includes a top frame disposed to allow the X-rays to be incident thereon and a side frame coupled to the top frame to define a side appearance of the X-ray detector. The middle block comprises an edge surface facing the side frame. The impact path is formed between the side frame and the edge surface.

The impact path extends from at least one of the side frame and the edge surface.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates a perspective view of an X-ray imaging apparatus according to an embodiment;

FIG. 2 illustrates operating principles of a sensor panel of the X-ray imaging apparatus;

FIG. 3 illustrates a perspective view of an X-ray detector according to a first exemplary embodiment;

FIG. 4 illustrates an exploded perspective view of the X-ray detector according to the first exemplary embodiment;

FIG. 5 illustrates a cross-sectional view of the X-ray detector of FIG. 3 taken along ling C-C′;

FIG. 6 illustrates a cross-sectional view illustrating an X-ray detector according to a second exemplary embodiment;

FIG. 7 illustrates a cross-sectional view of an X-ray detector according to a third exemplary embodiment;

FIG. 8 illustrates a cross-sectional view of an X-ray detector according to a fourth exemplary embodiment;

FIG. 9 illustrates a cross-sectional view of an X-ray detector according to a fifth exemplary embodiment;

FIG. 10 illustrates a cross-sectional view of an X-ray detector according to a sixth exemplary embodiment;

FIG. 11 illustrates a cross-sectional view of an X-ray detector according to a seventh exemplary embodiment;

FIG. 12 illustrates a cross-sectional view of an X-ray detector according to an eighth exemplary embodiment; and

FIG. 13 illustrates a cross-sectional view of an X-ray detector according to a ninth exemplary embodiment.

### DETAILED DESCRIPTION

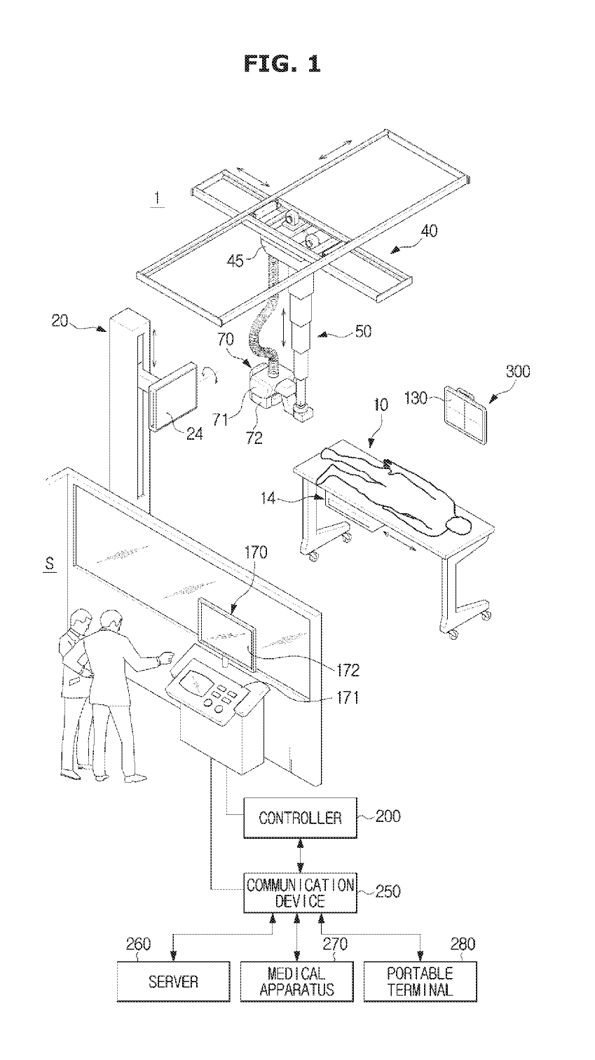

FIGS. 1 through 13, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged system or device.

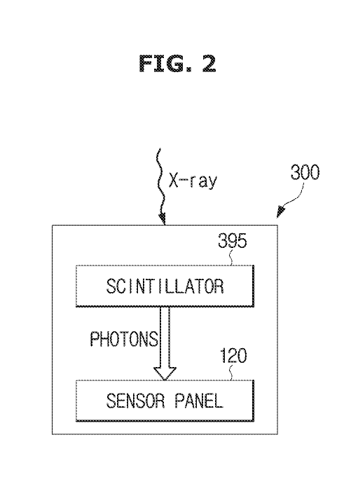

FIGS. 1 and 2 illustrate a case in which an X-ray detector 300 according to a first exemplary embodiment is used by way of example.

FIG. 1 illustrates a perspective view of an X-ray imaging apparatus according to an embodiment. FIG. 2 illustrates operating principles of a sensor panel of the X-ray imaging apparatus.

As illustrated in FIG. 1, an X-ray imaging apparatus 1 includes an X-ray source 70 configured to generate X-rays and emit the generated X-rays, an X-ray detector 300 configured to detect X-rays emitted from the X-ray source 70 and passing through a target object, and a workstation 170 configured to receive a command from a user and provide information. The X-ray imaging apparatus 1 may further include an imaging table 10 and an imaging stand 20 on which the X-ray detectors 300 are mounted. The X-ray imaging apparatus 1 may further include a controller 200 configured to control the X-ray imaging apparatus 1 in accordance with an input command and a communication device 250 configured to communicate with external devices.

The X-ray source 70 is a device emitting X-rays toward a target object. In this regard, the target object may be a living body of a human being or animal without being limited thereto and may also be any object whose internal structure may be imaged by the X-ray imaging apparatus 1.

The X-ray source 70 may include an X-ray tube 71 configured to generate X-rays and a collimator 72 configured to guide the generated X-rays toward the target object.

A guide rail 40 may be installed on the ceiling of an examination room in which the X-ray imaging apparatus 1 is located. The X-ray source 70 may move to a position corresponding to the target object in a state of being connected to a moving carriage 45 that moves along the guide rail 40. Since the moving carriage 45 is connected to the X-ray source 70 via a foldable post frame 50, a height of the X-ray source 70 may be adjusted.

The workstation 170 may be provided in a separate space S where X-rays are blocked and may be connected to the X-ray source 70 and the X-ray detector 300 via a wired or wireless communication network.

The workstation 170 may be provided with an input unit 171 to receive a command of the user and a display 172 to display information.

The input unit 171 may receive a command for imaging protocols, imaging conditions, imaging timing, position control of the X-ray source 70, and the like. The input unit 171 may include a keyboard, a mouse, a touch screen, a speech recognizer, and the like.

The display 172 may display a screen to guide the input of the user, an X-ray image, a screen indicating a state of the X-ray imaging apparatus 1, and the like.

The controller 200 may control imaging timing, imaging conditions, and the like in accordance with the command input by the user and create a medical image by using image data received from the X-ray detector 300. Also, the controller 200 may control positions of the X-ray source 70 or mounting parts 14 and 24 on which the X-ray detector 300 is mounted in accordance with the imaging protocols and a position of the target object.

The X-ray imaging apparatus 1 may be connected to an external device (such as external server 260, medical apparatus 270, portable terminal 280, smartphone, tablet PC, and wearable device) via the communication device 250 to transmit and receive data.

Meanwhile, the X-ray detector 300 may be implemented as a fixed type X-ray detector fixed to the imaging stand 20 or the imaging table 10 or a portable X-ray detector detachably mounted on the mounting parts 14 and 24 or used at a predetermined position. The portable X-ray detector may be implemented as a wired type or wireless type in accordance with data transmission methods and power supply methods.

A sub-user interface to provide the user with information and receive a command from the user may be disposed at one side of the X-ray source 70. Some or all of the functions performed by the input unit 171 and the display 172 of the workstation 170 may be performed by the sub-user interface.

Although FIG. 1 illustrates a fixed type X-ray imaging apparatus connected to the ceiling of the examination room, the X-ray imaging apparatus may also include various other X-ray imaging apparatuses such as a C-arm type X-ray imaging apparatus and a mobile X-ray imaging apparatus which are obvious to one of ordinary skill in the art.

The X-ray detector 300 is a device to detect X-rays that have passed through the target object. An incidence surface 130 on which X-rays are incident may be provided on a front surface of the X-ray detector 300, and a sensor panel 120 (FIG. 2) may be provided inside the X-ray detector 300.

The X-ray source 70 is a device to generate X-rays and emit the X-rays toward the target object and may include the X-ray tube 71 to generate X-rays.

The X-ray detector 300 is a device to detect X-rays that have been emitted from the X-ray source 70 and passed through the target object and detection of X-rays may be performed by the sensor panel 120 disposed in the X-ray detector 300. In addition, the sensor panel 120 converts the detected X-rays into electrical signals to acquire an image of the inside of the target object.

The sensor panel 120 may be classified according to a composition method thereof, a method of converting detected X-rays into electrical signals, and a method of acquiring the electrical signal.

First, the sensor panels 120 may be classified into a sensor panel including a monolithic type device and a sensor panel including a hybrid type device according to the composition method thereof.

When the sensor panel 120 includes the monolithic type device, one part to detect X-rays and generate an electrical signal and another part to read and process the electrical signal include semiconductors of the same material or are manufactured using the same process. For example, a light receiving device such as charge coupled device (CCD) or complementary metal oxide semiconductor (CMOS) may be used uniformly.

When the sensor panel 120 includes the hybrid type device, one part to detect X-rays and generate an electrical signal and another part to read and process the electrical signal include respective different materials or are manufactured using different processes. For example, a light receiving device such as a photodiode, a CCD, or CdZnTe may detect X-rays and a CMOS read out integrated circuit (CMOS ROIC) may read and process an electrical signal. For example, a strip detector may detect X-rays and the CMOS ROIC may read and process the electrical signal. Furthermore, for example, an a-Si or a-Se flat panel system may be used.

In addition, the sensor panel 120 may be classified into a direct conversion type and an indirect conversion type according to the method of converting X-rays into electrical signals.

According to the direct conversion type, when X-rays are emitted, electron-hole pairs are temporarily generated in a light receiving device, and electrons are moved toward a positive electrode and holes are moved toward a negative electrode according to an electric field applied between opposite ends of the light receiving device. In this case, the sensor panel 120 converts this movement into electrical signals. Examples of materials used for the light receiving device in the direct conversion type may include a-Se, CdZnTe, HgI₂, PbI₂, and the like.

As illustrated in FIG. 2, according to the indirect conversion type, when X-rays emitted from the X-ray source 70 (FIG. 1) react with a scintillator 395 and photons having a visible light wavelength are emitted, a light receiving device detects the photons and converts the photons into an electric signal. In the indirect conversion type, the light receiving device may include a-Si or the like, and a thin film type GADOX scintillator, a micro column type scintillator, a needle structure type CS (T1) scintillator, or the like may be used as the scintillator 395. In FIG. 2, the sensor panel 120 is used as a light receiving device.

In addition, the sensor panel 120 may be classified into a charge integration mode type to store electric charges for a predetermined period of time and acquire a signal therefrom and a photon counting mode type to count photons having energy equal to or greater than threshold energy when a signal is generated by a single X-ray photon, according to the method of acquiring an electrical signal.

The sensor panel 120 may be applied as any one of the above-described types.

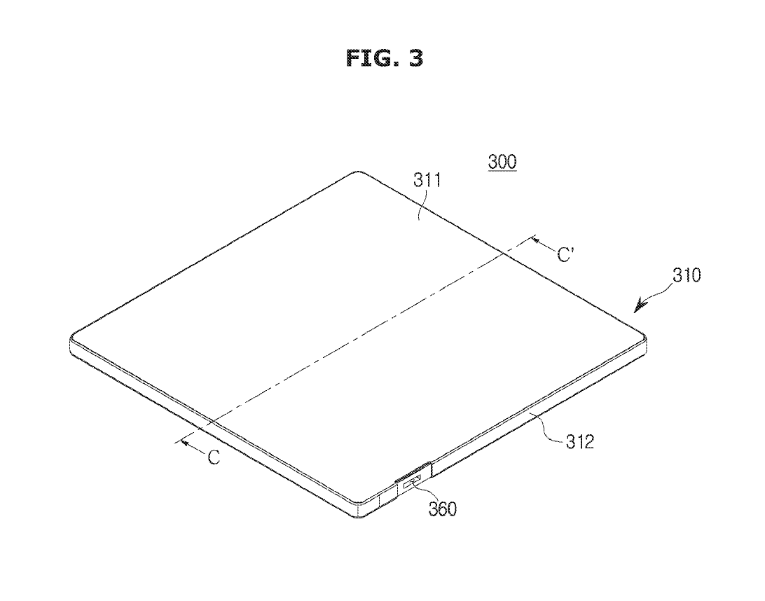

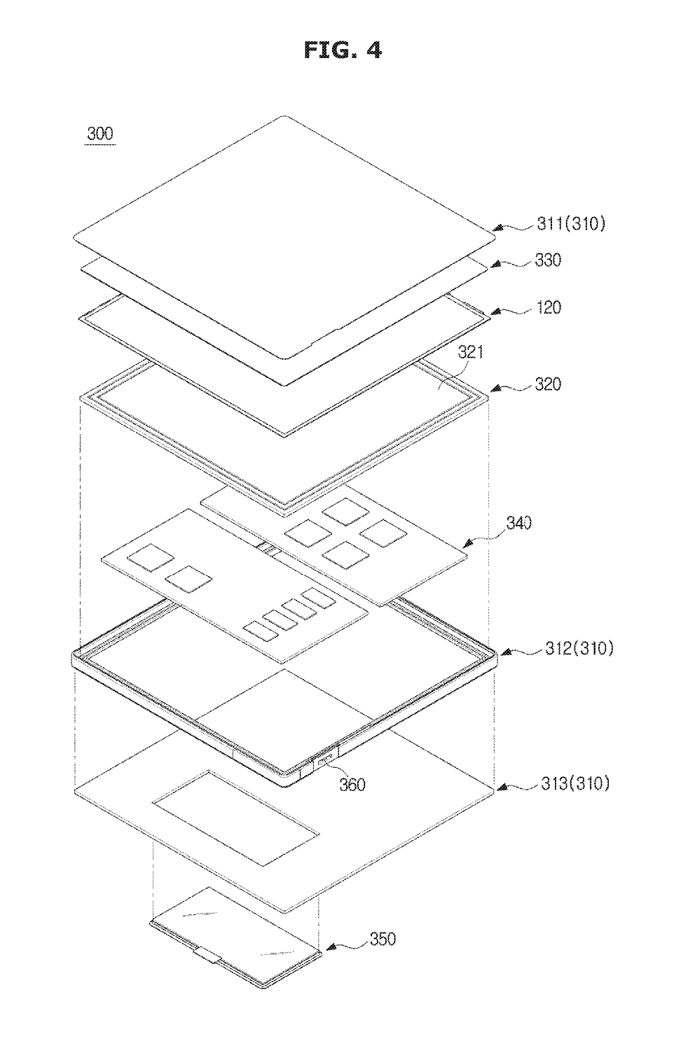

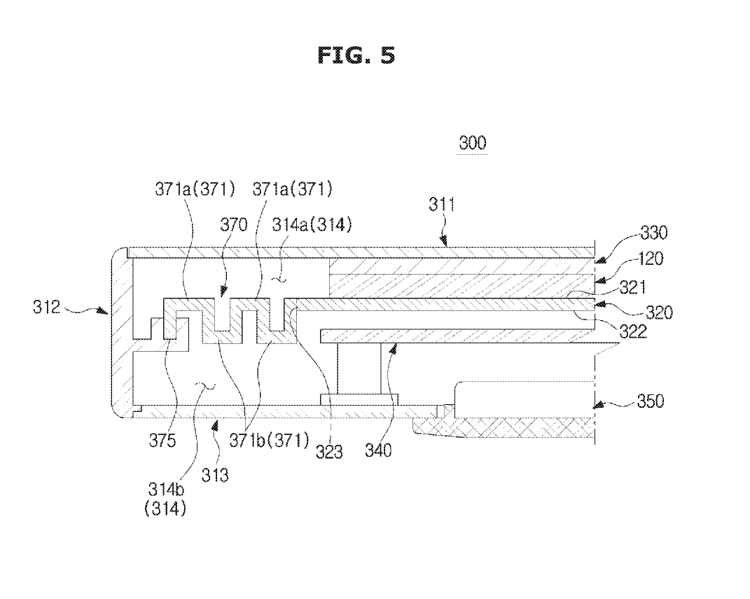

FIG. 3 illustrates a perspective view of an X-ray detector according to a first exemplary embodiment. FIG. 4 illustrates an exploded perspective view of the X-ray detector according to the first exemplary embodiment. FIG. 5 illustrates a cross-sectional view of the X-ray detector of FIG. 3 taken along ling C-C′.

As illustrated in FIGS. 3 to 5, the X-ray detector 300 may be provided to detect X-rays irradiated from the X-ray source 70.

The X-ray detector 300 may include a main body 310. The main body 310 may define an appearance of the X-ray detector 300.

The main body 310 may include a top frame 311. The top frame 311 may be disposed such that X-rays emitted by the X-ray source 70 are incident thereon. In other words, the top frame 311 may be provided with the incidence surface 130 (FIG. 1). The top frame 311 may define a top appearance of the X-ray detector 300. The top frame 311 may be provided in the form of a carbon plate or the like. The top frame 311 may further be provided with a deco sheet (not shown).

The main body 310 may further include a side frame 312. The side frame 312 may be coupled to the top frame 311 to define a side appearance of the X-ray detector 300. The side frame 312 may connect the top frame 311 with a bottom frame 313.

The main body 310 may further include the bottom frame 313. The bottom frame 313 may be disposed to face the top frame 311. The bottom frame 313 may define a bottom appearance of the X-ray detector 300.

The main body 310 may be formed by coupling of the top frame 311, the side frame 312, and the bottom frame 313.

The main body 310 may further have an internal space 314. The internal space 314 may be partitioned into a first space 314a and a second space 314b by a middle block 320 and a buffer member 370. The top frame 311 may define the first space 314a and the bottom frame 313 may define the second space 314b. The sensor panel 120 which will be described later may be disposed in the first space 314a. A circuit board 340 and a battery 350 which will be described later may be disposed in the second space 314b.

The X-ray detector 300 may further include the sensor panel 120 disposed in the main body 310 to convert X-rays emitted from the X-ray source 70 into electrical signals.

The sensor panel 120 may be disposed on the middle block 320. That is, the sensor panel 120 may be supported by the middle block 320.

The X-ray detector 300 may further include a scintillator (as shown in FIG. 2).

The scintillator may include a fluorescent material. The scintillator may convert incident X-rays into visible light. A cover (not shown) to protect the scintillator may be disposed on one surface of the scintillator. The scintillator may be formed of a metal such as aluminum.

The X-ray detector 300 may further include the middle block 320.

The middle block 320 may be disposed inside the main body 310 to support the sensor panel 120.

The middle block 320 may have a first surface 321 facing the top frame 311. The sensor panel 120 may be mounted on the first surface 321 of the middle block 320.

The middle block 320 may further include a second surface 322 facing the bottom frame 313.

The middle block 320 may further include an edge surface 323 facing the side frame 312.

The X-ray detector 300 may further include at least one cushion member 330. The at least one cushion member 330 may be disposed inside the main body 310 to prevent external impact from being transmitted to the sensor panel 120. For example, the at least one cushion member 330 may be disposed between the top frame 311 and the sensor panel 120. The at least one cushion member 330 prevents the sensor panel 120 from being damaged or destroyed by an external impact applied to the top frame 311. The at least one cushion member 330 may include an elastic material. For example, the at least one cushion member 330 may be formed of rubber, silicon, or the like. Also, the at least one cushion member 330 may be implemented as a sponge.

The X-ray detector 300 may further include a circuit board 340.

The circuit board 340 performs computation to acquire an image of the target object by using data acquired based on signals read by the sensor panel 120. The circuit board 340 may be accommodated in the X-ray detector 300 to control operation of the X-ray detector 300. That is, the circuit board 340 may be accommodated inside the main body 310.

The circuit board 340 may be disposed in the main body 310 to be spaced apart from the middle block 320. Particularly, the circuit board 340 may be disposed above the bottom frame 313 to be spaced apart from the middle block 320. By disposing the circuit board 340 to be spaced apart from the middle block 320 as described above, impact energy transmitted to the sensor panel 120 via the middle block 320 may be reduced when the middle block 320 and the side frame 312 collide. That is, when the circuit board 340 is coupled to the middle block 320, a total mass of the middle block 320 increases, and thus impact energy transmitted to the sensor panel 120 via the middle block 320 may increase in case of collision between the middle block 320 and the side frame 312. On the contrary, since the total mass of the middle block 320 decreases by coupling the circuit board 340 to the bottom frame 313 to be spaced apart from the middle block 320, the impact energy transmitted to the sensor panel 120 via the middle block 320 may decrease in case of collision between the middle block 320 and the side frame 312.

The circuit board 340 may include a memory and a computation device. The memory may store shadow information of the target object in accordance with irradiation angles of X-rays, and the computation device may calculate the irradiation angles of X-rays based on a shadow shape of the target object formed on the sensor panel 120 and the shadow information of the memory. The memory and the computation device may also be disposed outside the X-ray detector 300.

The sensor panel 120 and the circuit board 340 may be electrically connected to each other.

The X-ray detector 300 may further include the battery 350. The battery 350 may be accommodated inside the main body 310 to supply power to the circuit board 340.

The battery 350 may be disposed inside the main body 310 to be spaced apart from the middle block 320. Particularly, the battery 350 may be disposed on the bottom frame 313 to be spaced apart from the middle block 320. By disposing the battery 350 to be spaced apart from the middle block 320 as described above, impact energy transmitted to the sensor panel 120 via the middle block 320 may be reduced when the middle block 320 collides with the side frame 312.

For example, the battery 350 may be disposed on the bottom frame 313 to be located between the circuit board 340 and the bottom frame 313.

The X-ray detector 300 may further include a terminal unit 360 to which a coupling module (not shown) is coupled. The terminal unit 360 may be disposed at the X-ray detector 300 to be coupled to the coupling module. In other words, the terminal unit 360 may be disposed at the X-ray detector 300 such that the coupling module that is electrically connected to the circuit board 340 is coupled thereto. That is, the coupling module may be electrically connected to the circuit board 340 by being coupled to the terminal unit 360. Particularly, the terminal unit 360 may be formed at the side frame 312 of the main body 310.

The X-ray detector 300 may further include the buffer member 370. The buffer member 370 may protect the sensor panel 120 from impact. Particularly, the buffer member 370 may protect the sensor panel 120 from an impact applied to the side frame 312 or an impact caused by a collision between the side frame 312 and the middle block 320.

The buffer member 370 may extend from at least one of the main body 310 and the middle block 320 to have at least one bent portion 371. Particularly, the buffer member 370 may extend from at least one of the side frame 312 or an edge surface 323 of the middle block 320. According to another aspect, the buffer member 370 may extend from at least one of the side frame 312 and the edge surface 323 of the middle block 320 to have a wrinkled portion.

The side frame 312 and the middle block 320 may be coupled to each other via the buffer member 370.

According to another aspect, the X-ray detector 300 may further include an impact path 380 (FIG. 5). The impact path 380 may be formed between the main body 310 and the middle block 320 such that an impact applied to the main body 310 moves. The impact path 380 may include at least one bent portion 371 to reduce the impact applied to the main body 310. Particularly, the impact path 380 may be formed between the side frame 312 and the edge surface 323 of the middle block 320. The impact path 380 may extend from at least one of the side frame 312 and the edge surface 323 of the middle block 320. In this regard, the impact path 380 may be used as a concept including the buffer member 370.

As illustrated in FIGS. 3 to 5, the X-ray detector 300 may include the buffer member 370 that extends from the middle block 320 to have at least one bent portion 371. Particularly, the buffer member 370 may extend from the edge surface 323 of the middle block 320 toward the outside of the X-ray detector 300 to have at least one bent portion 371. In this case, the buffer member 370 may be integrally formed with the middle block 320.

The at least one bent portion 371 may include a first bent portion 371a convex toward the top frame 311 and a second bent portion 371b convex toward the bottom frame 313.

The buffer member 370 may include at least one bent portion 371 such that the first bent portion 371a and the second bent portion 371b are alternately aligned.

Intervals between the first bent portions 371a and the second bent portions 371b may be the same or different.

The side frame 312 may be provided with a coupling part 375 for coupling with the middle block 320. Particularly, the coupling part 375 may be provided at the inner surface of the side frame 312 such that the buffer member 370 extending from the edge surface 323 of the middle block 320 is coupled thereto. The coupling part 375 may be integrally formed with the side frame 312.

One end of the buffer member 370 facing the outside of the X-ray detector 300 may be coupled to the side frame 312. Particularly, the one end of the buffer member 370 facing the outside of the X-ray detector 300 may be coupled to the coupling part 375 of the side frame 312.

Since an impact applied to the X-ray detector 300 is reduced while passing through the at least one bent portion 371, the impact transferred to the sensor panel 120 may be minimized.

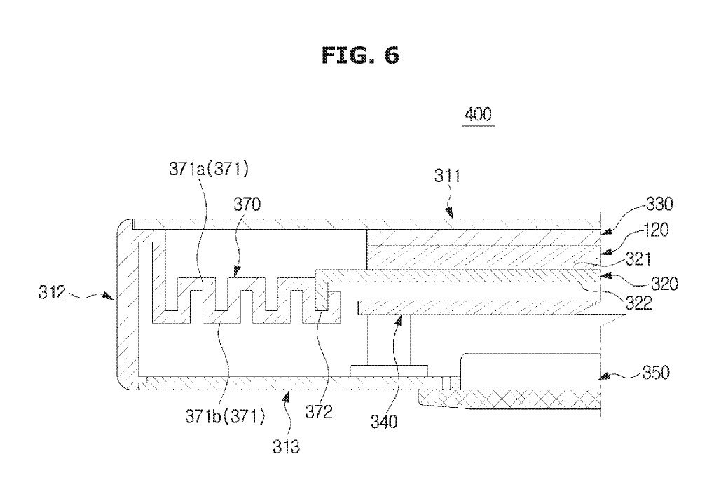

FIG. 6 illustrates a cross-sectional view of an X-ray detector according to a second exemplary embodiment. Hereinafter, descriptions given above with reference to the X-ray detector 300 according to the first exemplary embodiment will not be repeated.

As illustrated in FIG. 6, an X-ray detector 400 may include a buffer member 370 extending from the side frame 312 to have at least one bent portion 371. Particularly, the buffer member 370 may extend from the side frame 312 toward the inside of the X-ray detector 400. In this case, the buffer member 370 may be integrally formed with the side frame 312.

The at least one bent portion 371 may include a first bent portion 371a convex toward the top frame 311 and a second bent portion 371b convex toward the bottom frame 313.

The buffer member 370 may include at least one bent portion 371 such that the first bent portion 371a and the second bent portion 371b are alternately aligned.

Intervals between the first bent portions 371a and the second bent portions 371b may be the same or different.

The buffer member 370 may further include a fastening part 372 to couple the side frame 312 with the middle block 320. Particularly, the fastening part 372 may be provided at one end of the buffer member 370 facing the inside of the X-ray detector 400. The middle block 320 may be coupled to the fastening part 372. An edge portion of the middle block 320 may have a bent shape so as to be fitted into the fastening part 372.

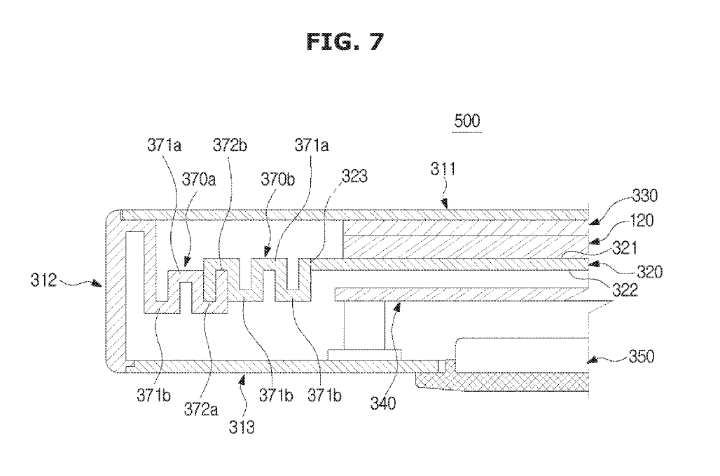

FIG. 7 illustrates a cross-sectional view of an X-ray detector according to a third exemplary embodiment. Hereinafter, descriptions given above with reference to the X-ray detector 300 according to the first exemplary embodiment will not be repeated.

As illustrated in FIG. 7, an X-ray detector 500 may include a first buffer member 370a extending from the side frame 312 to have at least one first bent portion 371a and 371b and a second buffer member 370b extending from the edge surface 323 of the middle block 320 to have at least one second bent portion 371a and 371b. In this case, the first buffer member 370a may extend from the side frame 312 toward the inside of the X-ray detector 500 and may be integrally formed with the side frame 312. In addition, the second buffer member 370b may extend from the edge surface 323 of the middle block 320 toward the outside of the X-ray detector 500 and may be integrally formed with the middle block 320.

Since the at least one first bent portion 371a and 371b and the at least one second bent portion 371a and 371b are described above with reference to the X-ray detector 300 according to the first exemplary embodiment and the X-ray detector 400 according to the second exemplary embodiment, detailed descriptions thereof will not be repeated. Particularly, the at least one first bent portion 371a and 371b and the at least one second bent portion 371a and 371b of FIG. 7 are concepts including at least one first bent portion 371a and at least one second bent portion 371b, respectively.

At least one of the first buffer member 370a and the second buffer member 370b may include the fastening part 372 to couple the side frame 312 with the middle block 320. For example, the first buffer member 370a and the second buffer member 370b may include a first fastening part 372a and a second fastening part 372b, respectively, to couple the side frame 312 with the middle block 320. The first fastening part 372a may be disposed at one end portion of the first buffer member 370a facing the inside of the X-ray detector 500. The second fastening part 372b may be disposed at one portion of the second buffer member 370b facing the outside of the X-ray detector 500. The first fastening part 372a and the second fastening part 372b may be fitted into each other, and thus the side frame 312 and the middle block 320 may be coupled with each other.

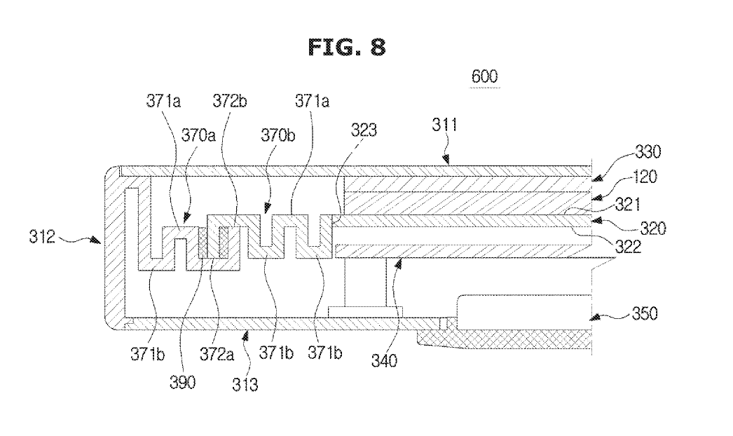

FIG. 8 illustrates a cross-sectional view of an X-ray detector according to a fourth exemplary embodiment. Hereinafter, descriptions given above with reference to the X-ray detector 300 according to the first exemplary embodiment, the X-ray detector 400 according to the second exemplary embodiment, and the X-ray detector 500 according to the third exemplary embodiment will not be repeated.

As illustrated in FIG. 8, an X-ray detector 600 may further include an impact absorbing member 390. The impact absorbing member 390 may effectively protect the sensor panel 120 from an impact applied to the X-ray detector 600 by assisting the buffer member 370. The impact absorbing member 390 may be disposed to prevent direct contact between the side frame 312 and the middle block 320. Particularly, the impact absorbing member 390 may be disposed to prevent direct contact between the first fastening part 372a and the second fastening part 372b. The impact absorbing member 390 may be disposed at one of the first fastening part 372a and the second fastening part 372b.

The impact absorbing member 390 may include an elastic material. For example, the impact absorbing member 390 may be implemented using rubber, silicon, urethane, and the like. In addition, the impact absorbing member 390 may be implemented using sponge.

As described above, the impact absorbing member 390 may effectively block an impact that may be transmitted to the middle block 320 via the side frame 312 together with the buffer member 370.

In case of the X-ray detector 300 according to the first exemplary embodiment, the impact absorbing member 390 may be disposed at the coupling part 375.

In the X-ray detector 400 according to the second exemplary embodiment and the X-ray detector 500 according to the third exemplary embodiment, the impact absorbing member 390 may be disposed at the fastening part 372.

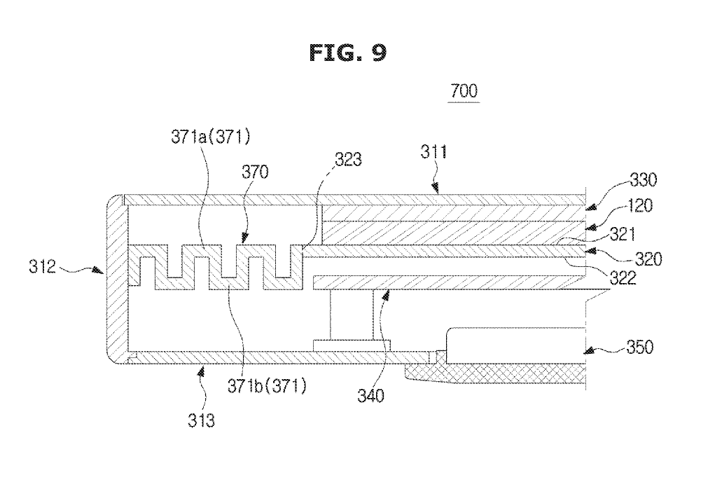

FIG. 9 illustrates a cross-sectional view of an X-ray detector according to a fifth exemplary embodiment. Hereinafter, descriptions given above with reference to the X-ray detector 300 according to the first exemplary embodiment will not be repeated.

As illustrated in FIG. 9, an X-ray detector 700 may include a buffer member 370 extending from the middle block 320 to have at least one bent portion 371. Particularly, the buffer member 370 may extend from the edge surface 323 of the middle block 320 toward the outside of the X-ray detector 700 to have at least one bent portion 371. In this case, the buffer member 370 may be integrally formed with the middle block 320.

One end of the buffer member 370 facing the outside of the X-ray detector 700 may be fixedly coupled to the inner surface of the side frame 312.

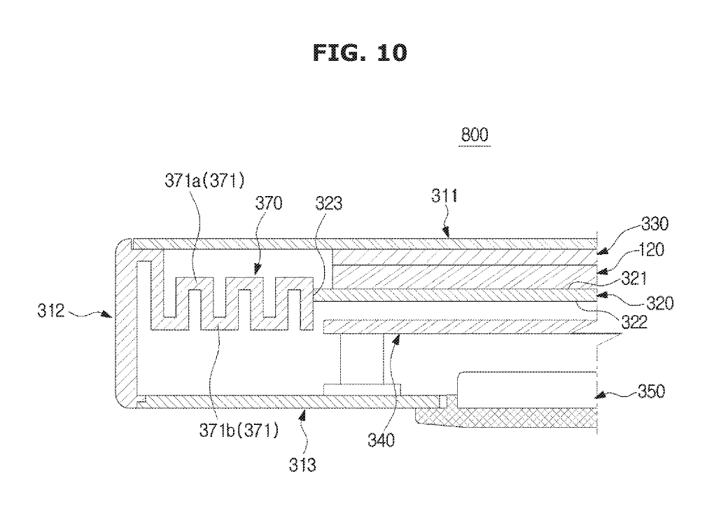

FIG. 10 illustrates a cross-sectional view of an X-ray detector according to a sixth exemplary embodiment. Hereinafter, descriptions given above with reference to the X-ray detector 300 according to the first exemplary embodiment will not be repeated.

As illustrated in FIG. 10, an X-ray detector 800 may include a buffer member 370 extending from the side frame 312 to have at least one bent portion 371. Particularly, the buffer member 370 may extend from the side frame 312 toward the inside of the X-ray detector 800. In this case, the buffer member 370 may be integrally formed with the side frame 312.

One end of the buffer member 370 facing the inside of the X-ray detector 800 may be fixedly coupled to the edge surface 323 of the middle block 320.

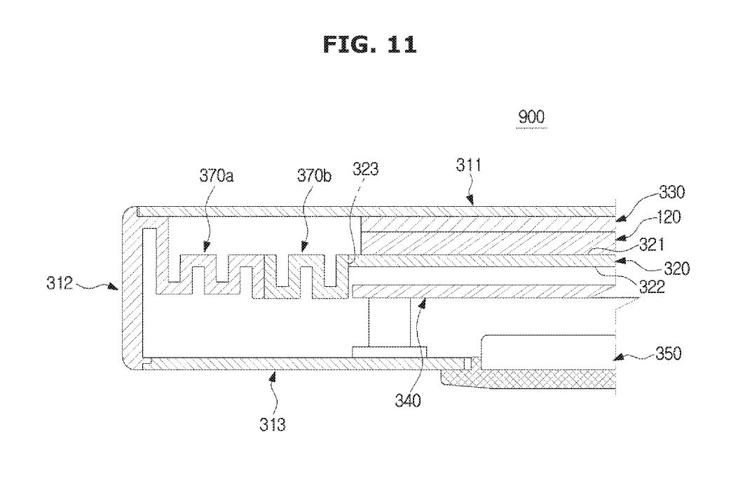

FIG. 11 illustrates a cross-sectional view of an X-ray detector according to a seventh exemplary embodiment. Hereinafter, descriptions given above with reference to the X-ray detector 300 according to the first exemplary embodiment will not be repeated.

As illustrated in FIG. 11, an X-ray detector 900 may include a first buffer member 370a extending from the side frame 312 to have at least one first bent portion 371a and 371b and a second buffer member 370b extending from the edge surface 323 of the middle block 320 to have at least one second bent portion 371a and 371b. In this case, the first buffer member 370a may extend from the side frame 312 toward the inside of the X-ray detector 900 and may be integrally formed with the side frame 312. In addition, the second buffer member 370b may extend from the edge surface 323 of the middle block 320 toward the outside of the X-ray detector 900 and may be integrally formed with the middle block 320.

One end of the first buffer member 370a facing the inside the X-ray detector 900 and one end of the second buffer member 370b facing the outside of the X-ray detector 900 may be fixedly coupled to each other.

The at least one first bent portion 371a and 371b and the at least one second bent portion 371a and 371b of FIG. 11 are concepts including at least one first bent portion 371a and at least one second bent portion 371b, respectively.

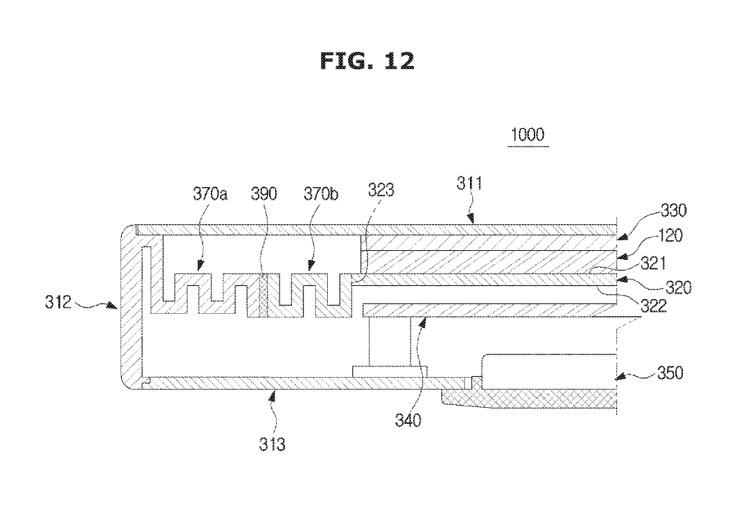

FIG. 12 illustrates a cross-sectional view of an X-ray detector according to an eighth exemplary embodiment. Hereinafter, descriptions given above with reference to the X-ray detector 300 according to the first exemplary embodiment, the X-ray detector 700 according to the fifth exemplary embodiment, the X-ray detector 800 according to the sixth exemplary embodiment, and the X-ray detector 900 according to the seventh exemplary embodiment will not be repeated.

As illustrated in FIG. 12, an X-ray detector 1000 may further include an impact absorbing member 390. The impact absorbing member 390 may effectively protect the sensor panel 120 from an impact applied to the X-ray detector 1000 by assisting the buffer member 370. The impact absorbing member 390 may be disposed to prevent direct contact between the side frame 312 and the middle block 320. Particularly, the impact absorbing member 390 may be disposed to prevent direct contact between the first buffer member 370a and the second buffer member 370b. The impact absorbing member 390 may be disposed between one end of the first buffer member 370a facing the inside of the X-ray detector 1000 and one end of the second buffer member 370b facing the outside of the X-ray detector 1000.

The impact absorbing member 390 may include an elastic material. For example, the impact absorbing member 390 may be implemented using rubber, silicon, urethane, and the like. In addition, the impact absorbing member 390 may be implemented using sponge.

As described above, the impact absorbing member 390 may effectively block an impact that may be transmitted to the middle block 320 via the side frame 312 together with the buffer member 370.

In case of the X-ray detector 700 according to the fifth exemplary embodiment, the impact absorbing member 390 may be disposed between one end of the buffer member 370 facing the outside of the X-ray detector 700 and the inner surface of the side frame 312.

In case of the X-ray detector 800 according to the sixth exemplary embodiment, the impact absorbing member 390 may be disposed between one end of the buffer member 370 facing the inside of the X-ray detector 800 and the edge surface 323 of the middle block 320.

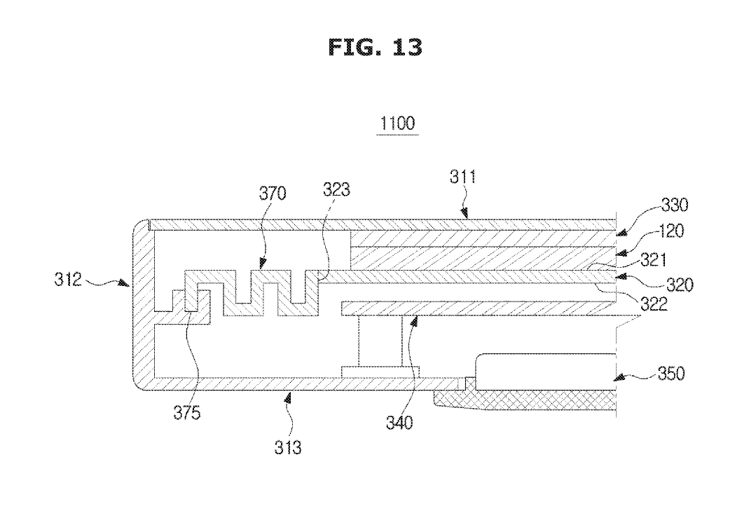

FIG. 13 illustrates a cross-sectional view of an X-ray detector according to a ninth exemplary embodiment. Hereinafter, descriptions given above with reference to the X-ray detector 300 according to the first exemplary embodiment will not be repeated.

As illustrated in FIG. 13, an X-ray detector 1100 may include a main body 310 in which the bottom frame 313 and the side frame 312 are integrally formed with each other.

According to another aspect, the main body 310 of the X-ray detector 1100 may have a monocoque structure. That is, the X-ray detector 1100 may become more lightweight and have improved impact resistance by designing the bottom frame 313 and the side frame 312 to have a monocoque structure. In addition, when the bottom frame 313 and the side frame 312 are designed to have the monocoque structure, manufacturing costs may be reduced by using a pressing process instead of a die casting process.

As is apparent from the above description, damage of the sensor panel caused by an external impact applied to the X-ray detector may be prevented by applying the buffer member having at least one bent portion thereto.

A buffer member structure may be simplified and the number of parts involved in the buffer membering operation may be reduced by using the buffer member extending from at least one of the side frame and the middle block and having at least one bent portion instead of using a separate buffer member member.

The sensor panel may efficiently be protected from external deformation of the X-ray detector caused by an external force by integrating the bottom frame with the side frame.

Impact energy transmitted to the sensor panel via the middle block may be reduced by disposing at least one of the battery and the circuit board to be spaced apart from the middle block.

Although the present disclosure has been described with an exemplary embodiment, various changes and modifications may be suggested to one skilled in the art. It is intended that the present disclosure encompass such changes and modifications as fall within the scope of the appended claims.

What is claimed is:

1. An X-ray imaging apparatus comprising:
an X-ray source configured to generate X-rays and emit the generated X-rays; and
an X-rays detector configured to detect the X-rays emitted by the X-ray source, wherein the X-ray detector comprises:
a main body;
a sensor panel disposed in the main body, the sensor panel is configured to convert the X-rays emitted by the X-ray source into electrical signals;
a middle block disposed in the main body, the middle block is configured to support the sensor panel; and
a buffer member extending between the main body and the middle block, the buffer member includes at least one bent portion.

2. The X-ray imaging apparatus according to claim 1, wherein the main body comprises:
a top frame configured to receive the X-rays emitted by the X-ray source; and
a side frame coupled to the top frame,
wherein the middle block comprises an edge surface facing the side frame, and
wherein the buffer member extends between the side frame and the edge surface.

3. The X-ray imaging apparatus according to claim 2, wherein the main body further comprises a bottom frame disposed to face the top frame, the bottom frame and the side frame are integrally formed with each other.

4. The X-ray imaging apparatus according to claim 2, wherein the side frame and the middle block are coupled to each other via the buffer member.

5. The X-ray imaging apparatus according to claim 2, wherein the buffer member comprises a fastening part configured to couple the side frame with the middle block.

6. The X-ray imaging apparatus according to claim 2, wherein the buffer member extends from the side frame to have the at least one bent portion, and one end of the buffer member is fixedly coupled to the edge surface.

7. The X-ray imaging apparatus according to claim 2, wherein the buffer member extends from the edge surface to have the at least one bent portion, and one end of the buffer member is fixedly coupled to an inner surface of the side frame.

8. The X-ray imaging apparatus according to claim 2, wherein the buffer member comprises:
a first buffer member extending from the side frame to have at least one first bent portion; and
a second buffer member extending from the edge surface to have at least one second bent portion,
wherein one end of the first buffer member and one end of the second buffer member are fixedly coupled to each other.

9. The X-ray imaging apparatus according to claim 2, wherein the buffer member comprises:
a first buffer member extending from the side frame to have at least one first bent portion; and
a second buffer member extending from the edge surface to have at least one second bent portion,
wherein the X-ray detector further comprises an impact absorbing member disposed between one end of the first buffer member and one end of the second buffer member facing the one end of the first buffer member.

10. The X-ray imaging apparatus according to claim 1, wherein the X-ray detector further comprises a circuit board configured to control operation of the X-ray detector, and
the circuit board is disposed in the main body and spaced apart from the middle block.

11. The X-ray imaging apparatus according to claim 10, wherein the X-ray detector further comprises a battery configured to supply power to the circuit board, and
the battery is disposed in the main body and spaced apart from the middle block.

12. An X-ray detector provided to detect X-rays comprising:
a main body having an internal space;
a middle block disposed in the internal space;
a buffer member extending from between the main body and the middle block, the buffer member includes at least one bent portion and is configured to partition the internal space into a first space and a second space together with the middle block; and
a sensor panel disposed in the first space, the sensor panel is configured to convert the X-rays into electrical signals.

13. The X-ray detector according to claim 12, further comprising a circuit board configured to control operation of the X-ray detector, the circuit board is disposed in the second space spaced apart from the middle block.

14. The X-ray detector according to claim 13, further comprising a battery configured to supply power to the circuit board, the battery is disposed in the second space and spaced apart from the middle block.

15. The X-ray detector according to claim 12, wherein the main body comprises:
a top frame configured to define the first space; and
a side frame extending in an alignment direction of the first space and the second space and coupled to the top frame,
wherein the middle block comprises an edge surface facing the side frame, and
the buffer member extends between the side frame and the edge surface.

16. The X-ray detector according to claim 15, wherein the main body further comprises a bottom frame configured to define the second space, the bottom frame and the side frame are integrally formed with each other.

17. The X-ray detector according to claim 15, wherein the side frame and the middle block are coupled to each other via the buffer member.

18. An X-ray detector provided to detect X-rays comprising:
a main body;
a sensor panel disposed in the main body, the sensor panel is configured to convert the X-rays into electrical signals;
a middle block disposed in the main body, the middle block is configured to support the sensor panel; and
an impact path disposed between the main body and the middle block, the impact path is configured to move an impact applied to the main body and comprises at least one bent portion configured to reduce the impact applied to the main body.

19. The X-ray detector according to claim 18, wherein the main body comprises:
a top frame configured to receive the X-rays; and
a side frame coupled to the top frame,
wherein the middle block comprises an edge surface facing the side frame, and
wherein the impact path is formed between the side frame and the edge surface.

20. The X-ray detector according to claim 19, wherein the impact path extends between the side frame and the edge surface.
